(19) United States
(12) Patent Application Publication
(10) Pub. No.: US 2003/0108990 A1
(43) Pub. Date: Jun. 12, 2003

(54) Method for preparing deletion mutants

(76) Inventors: Jorg Kamper, Amonenburg (DE); Peter Schreier, Koln (DE)

Correspondence Address: BAYER CORPORATION, 100 BAYER ROAD, PITTSBURGH, PA, 15205, US

(21) Appl. No.: 10/191,381

(22) Filed: Jul. 8, 2002

(30) Foreign Application Priority Data
Jul. 12, 2001 (DE) 10133928.3

(51) Int. Cl. C12P 21/02; C12P 19/34; C12N 1/18; C12N 1/16; C12N 15/74

(52) U.S. Cl. 435/69.1; 435/254.21; 435/254.1; 435/483; 435/91.2; 435/320.1

(57) ABSTRACT

The present invention provides a universally usable knockout cassette that contains a selectable marker gene and is characterized by the specific design of the restriction cleavage sites. A method for preparing the knockout cassette is also provided as are methods of using the knockout cassette.

### FIELD OF THE INVENTION

The present invention relates in general to methods for preparing deletion mutants based on polymerase chain reaction (PCR), and more specifically to a universally usable knockout cassette which contains a selectable marker gene and which is characterized by the specific design of the restriction cleavage sites therein.

### BACKGROUND OF THE INVENTION

The functional analysis of genes in many cases requires the deletion of the appropriate genes or the replacement thereof with modified genes. The traditional method for preparing deletion mutants, for example in the basidiomycete Ustilago maydis, is the transformation with linear deoxyribonucleic acid (“DNA”) fragments (Bölker M., Bohnert H. U., Braun K. H., Gorl J., Kahmann R. (1995). Tagging pathogenicity genes in Ustilago maydis by restriction enzyme-mediated integration (REMI), Mol. Gen. Genet. 248, 547-552.; Fotheringham S., Holloman W. K. (1989). Cloning and disruption of Ustilago maydis genes, Mol. Cell. Biol. 9, 4052-4055; Kronstad J. W., Wang J., Covert S. F., Holden D. W., McKnight G. L., Leong S. A. (1989). Isolation of metabolic genes and demonstration of gene disruption in the phytopathogenic fungus Ustilago maydis, Gene 79, 97-106; Tsukuda T., Carleton S., Fotheringham S., Holloman W. K. (1988). Isolation and characterization of an autonomously replicating sequence from Ustilago maydis, Mol. Cell. Biol. 8, 3703-3709; Wang J., Holden D. W., Leong S. A. (1988). Gene transfer system for the phytopathogenic fungus Ustilago maydis, Proc. Nat. Acad. Sci. USA 85, 865-869). These DNA fragments contain a selectable marker gene (e.g. hph, cbx, phleo) which is flanked by regions which are homologous towards the integration site. Experience shows that homologous recombination takes place via these flanking regions in 10% to 50% of transformants, and this fact can be used specifically for exchanging genes.

Knockout constructs for transformation are normally prepared via a plurality of time-consuming cloning steps in plasmid vectors (Basse C. W., Stumpferl S., Kahmann R. (2000). Characterization of a Ustilago maydis gene specifically induced during the biotrophic phase: evidence for negative as well as positive regulation, Mol. Cell. Biol. 20, 329-339; Lehmler C., Steinberg G., Snetselaar K. M., Schliwa M., Kahmann R., Bölker M. (1997). Identification of a motor protein required forfilamentous growth in Ustilago maydis, EMBO J. 16, 3464-3473; Quadbeck-Seeger C., Wanner G., Huber S., Kahmann R., Katmper J. (2000). A protein with similarity to the human retinoblastoma binding protein 2 acts specifically as a repressor for genes regulated by the b mating type locus in Ustilago maydis, Mol. Microbiol. 38,154-166; Regenfelder E., Spellig T., Hartmann A., Lauenstein S., Bölker M., Kahmann R. (1997). G proteins in Ustilago maydis: transmission of multiple signals? EMBO J. 16, 1934-1942). Because one must be certain that the regions required for recombination flank the relevant gene without overlapping with the open reading frame, several PCR steps are often required for constructing the knockout constructs. Prior to transformation into the genome of the organism to be studied, these plasmid vectors are normally linearized by a restriction enzyme which cuts in the plasmid backbone, i.e. outside the insert of interest.

The techniques known in the art for preparing knockout mutants have several disadvantages. Firstly, for each individual gene to be deleted a specific knockout construct has to be prepared in a plasmid vector. Secondly, this preparation method involves a multiplicity of time-consuming method steps, including linking the flanking regions of the gene of interest to the marker gene, ensuring that the flanking regions do not overlap with the open reading frame (ORF) of the gene to be knocked out and ensuring that the marker-gene ORF in the plasmid vector is intact. As a consequence of those disadvantages, it has heretofore not been possible to prepare deletion mutants in a high throughput process.

### SUMMARY OF THE INVENTION

Accordingly, the present invention obviates problems inherent in the art by providing a PCR-based method that produces a knockout construct which can be used directly for homologous recombination to generate deletion mutants. The knockout cassettes of the present invention can be used universally for any gene of interest.

The method of the present invention therefore provides the ability to prepare deletion mutants via a high throughput process. This is particularly important in methods for finding essential genes or those genes that are important for pathogenesis, such genes may be used to identify new targets for the search for active substances.

### BRIEF DESCRIPTION OF THE FIGURES

The present invention will now be described for purposes of illustration and not limitation in conjunction with the figures, wherein:

FIG. 1 provides a schematic representation of the method of the present invention in which,

Step A is the generation of the regions (white boxes) flanking a gene (grey bar) by PCR using the outer primers lb1/rb1 and the inner primers lb2/rb2. Primers lb2 and rb2 are extended beyond the flanking regions by two different cleavage sites for one restriction enzyme (here: Sfi I). (G=guanosine, C=cytosine, N=any base, abc=random sequence of three bases; def=sequence complementary to abc; uvw=random sequence of three bases, different from abc; xyz=sequence complementary to uvw);

Step B is the cleavage of the amplification products from step A by a restriction enzyme (here: Sfi I), resulting in two non-compatible overhanging ends;

Step C depicts the ligation of a knockout cassette (hatched bar) between the two flanking regions of the gene of interest, the knockout cassette has flanking region-compatible cleavage sites of the same restriction enzyme;

Step D is the amplification of the ligation product from step C via PCR using primers lb1 and rb1;

Step E is the transformation of the amplified ligation product from step D into a recipient cell;

Step F is the checking of transformation and successful homologous recombination by using whole-cell PCR with the primers test-lb/test-rb which in each case bind to regions outside the flanking regions chosen in step A in combination with the primers KK-lb and KK-rb which in each case bind to regions within the knockout cassette;

FIG. 2 provides a schematic representation of a knockout construct of the present invention in which F (white bar) represents the two flanking regions of the gene of interest, S (black square) represents the two different restriction cleavage sites, and M (hatched bar) represents the marker gene including promoter and terminator;

FIG. 3 provides schematic representations of three fragments which are combined to form the knockout cassette of the present invention. White bars in the three fragments indicate optional short DNA inserts of different length between the individual elements. R1 to R6 denote restriction cleavage sites with palindromic DNA sequence for six different restriction enzymes. RS-a and RS-b denote two different restriction cleavage sites with non-palindromic sequence for the same restriction enzyme;

FIG. 3A provides a schematic representation of fragment A containing marker gene (M) and terminator (T) and the restriction cleavage sites R1 to R4 and RS-b;

FIG. 3B provides a schematic representation of fragment B containing promoter (P) and the restriction cleavage sites R1, R5, R6 and RS-a;

FIG. 3C provides a schematic representation of fragment C containing the plasmid backbone (plasmid) DNA and the restriction cleavage sites R4 and R5;

FIG. 4 provides a schematic representation of the knockout construct of the present invention, wherein the same abbreviations are used as in FIG. 3;

FIG. 5 provides a physical map of plasmid pBS-hhn. The plasmid contains the truncated hsp70 promoter (hsp=heat shock protein) from Ustilago maydis and the hph gene (hygromycin-phosphotransferase gene) with the NOS terminator. The plasmid also contains eight cleavage sites for restriction enzymes: Not I (at nucleotide 957, from Nocardia otitidis-caviarum, sequence 5′-GC⇓GGCCGC-3′), Nco I (at nucleotide 1634 and 1988, sequence 5′-C⇓CATGG-3′), Eco RI (at nucleotide 1743, sequence 5′-G⇓AATTC-3′), Xho I (at nucleotide 2555, sequence 5′-C⇓TCGAG-3′), Kpn I (at nucleotide 2570, sequence 5′-GGTAC⇓C-3′), and at nucleotide 659 the cleavage site Sfi I-b and at nucleotide 2543 the cleavage site Sfi I-a, having the sequences indicated in the sequences listing;

FIG. 6A depicts the gel-electrophoretic fractionation of the PCR products for amplification of LB and RB (annealing temperature 60° C. and 65° C.) and of the Sfi I-cut vector pBS-hhn (LB=left flanking region of the b-locus gene, RB=right flanking region of the b-locus gene);

FIG. 6B depicts the gel-electrophoretic fractionation of the Sfi I-restricted and gel-purified flanking regions LB and RB and of the hph cassette;

FIG. 6C depicts the gel-electrophoretic fractionation of the ligation products;

FIG. 6D depicts the gel-electrophoretic fractionation after reamplification of bands A and B (from FIG. C) or of the entire ligation mixture;

FIG. 6E depicts the analysis of the crossing of 20 transformants each of mixture A and C with the strain FB2 (a2b2);

FIG. 6F shows the Southern analysis of 6 transformants each of mixtures A and C: genomic DNA of the transformants and of wild-type strain FB2 was cut with Sal I, fractionated by gel-electrophoresis and hybridized with the b locus as probe in a Southern blot analysis. Gene replacement results in a 2.1 kb band instead of a 5.9 kb band; and

FIG. 7 depicts the gel-electrophoretic fractionation of PCR products obtained using the primer combinations “KK-lb +lb1”, “KK-lb+test-lb”, “KK-rb+rb1” and “KK-rb+test-rb”. The DNA of 10 different transformants was used. Lane 1 contains1 kb molecular weight marker (sizes in kb). Lanes 2 to 6 correspond to the transformants analysed in lanes 1 to 5 in FIG. 6F. Lanes 7 to 11 correspond to the transformants analysed in lanes 7 to 11 in FIG. 6F. Lane 12 contains PCR reaction without addition of DNA.

### DETAILED DESCRIPTION OF THE INVENTION

The present invention will now be described for purposes of illustration and not limitation. The contents of all references are incorporated by reference in their entireties.

Information on the Sequence Listings

SEQ ID NO. 1: DNA sequence of primer lb1.

SEQ ID NO. 2: DNA sequence of primer lb2 with Sfi l cleavage site.

SEQ ID NO. 3: DNA sequence of primer rb1.

SEQ ID NO. 4: DNA sequence of primer rb2 with Sfi l cleavage site.

SEQ ID NO. 5: DNA sequence of primer KK-lb.

SEQ ID NO. 6: DNA sequence of primer KK-rb.

SEQ ID NO. 7: DNA sequence of primer test-lb.

SEQ ID NO. 8: DNA sequence of primer test-rb.

SEQ ID NO. 9: DNA sequence of the Sfi I-a cleavage site from plasmid pBS-hhn.

SEQ ID NO. 10: DNA sequence of the Sfi I-b cleavage site from plasmid pBS-hhn.

SEQ ID NO. 11: DNA sequence of primer hph-Nco/Bam.

SEQ ID NO. 12: DNA sequence of primer hph-STOP.

SEQ ID NO. 13: DNA sequence of primer 3-hph.

SEQ ID NO. 14: DNA sequence of primer 5-hsp.

SEQ ID NO. 15: DNA sequence of primer hsp-Nco.

SEQ ID NO. 16: DNA sequence of the Xho I/Sac I insert (hph knockout cassette) from plasmid pBS-hhn.

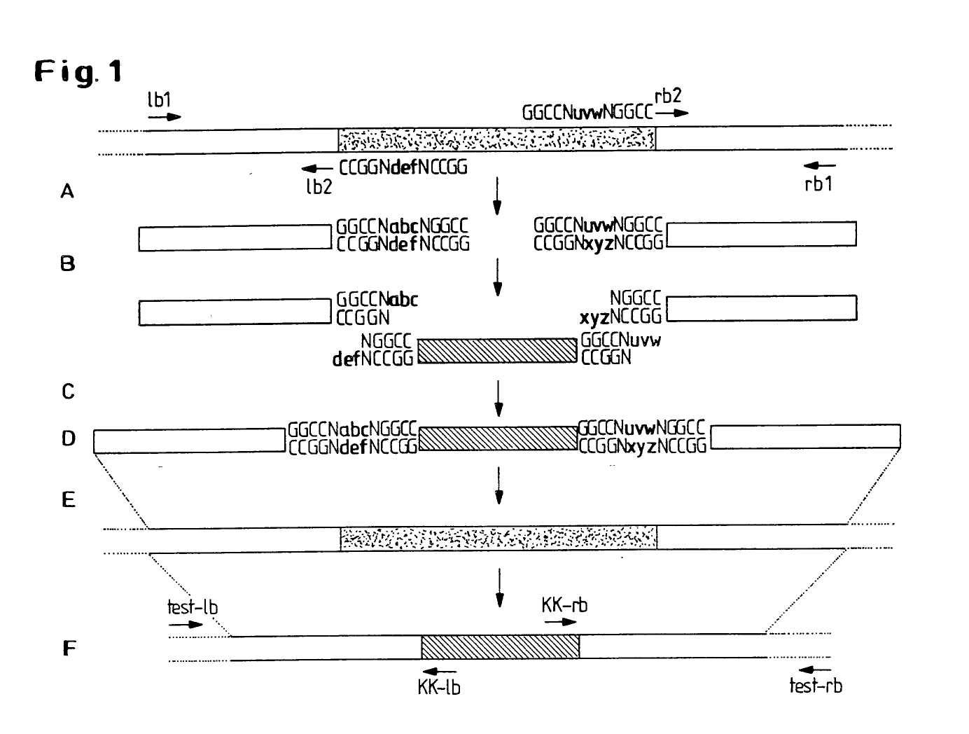

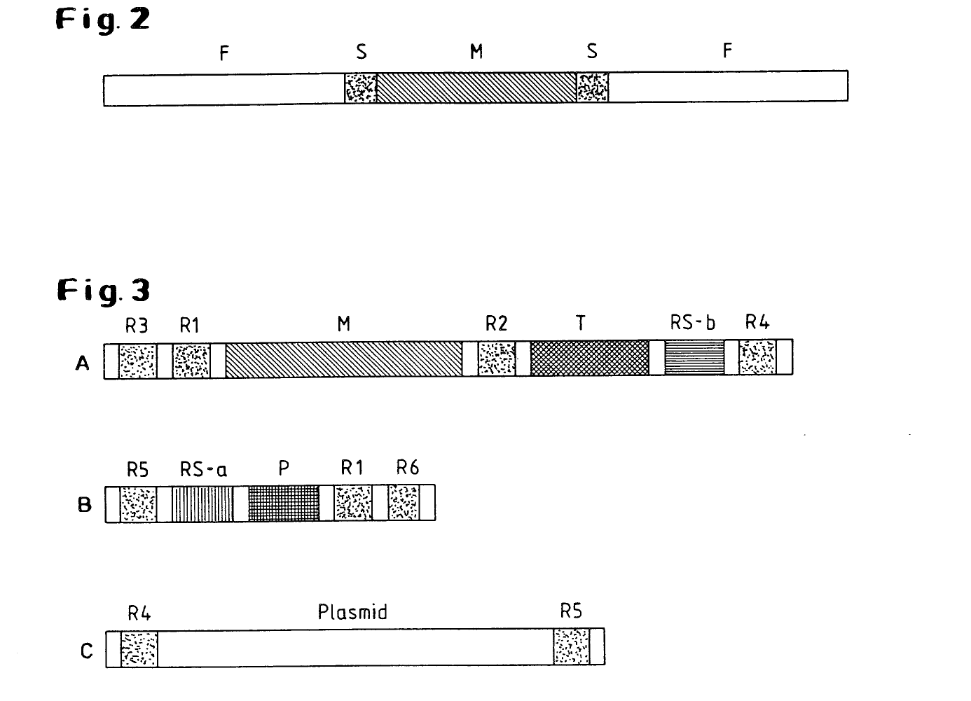

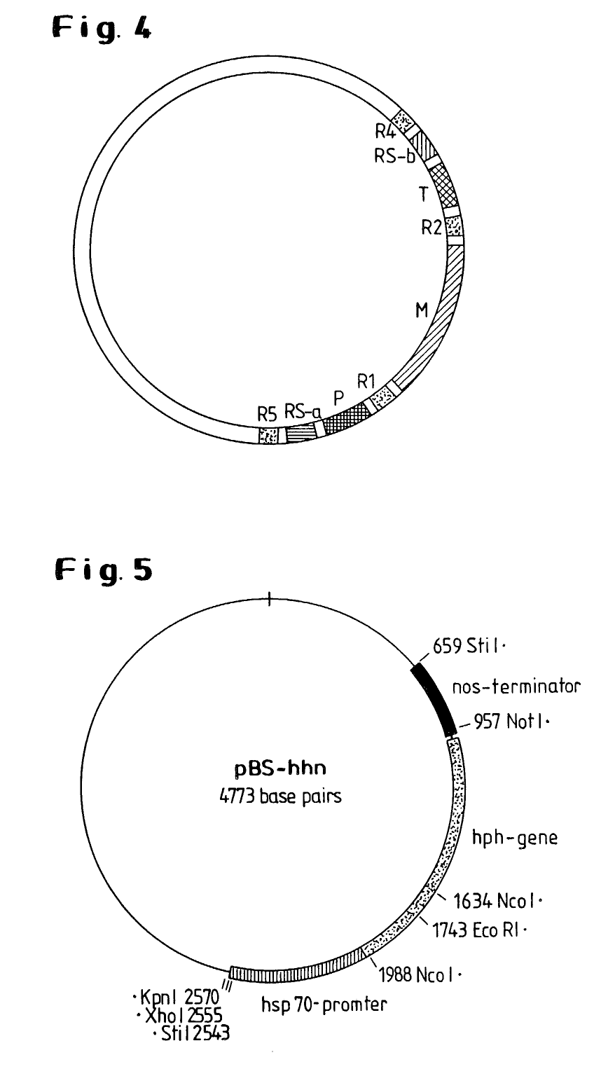

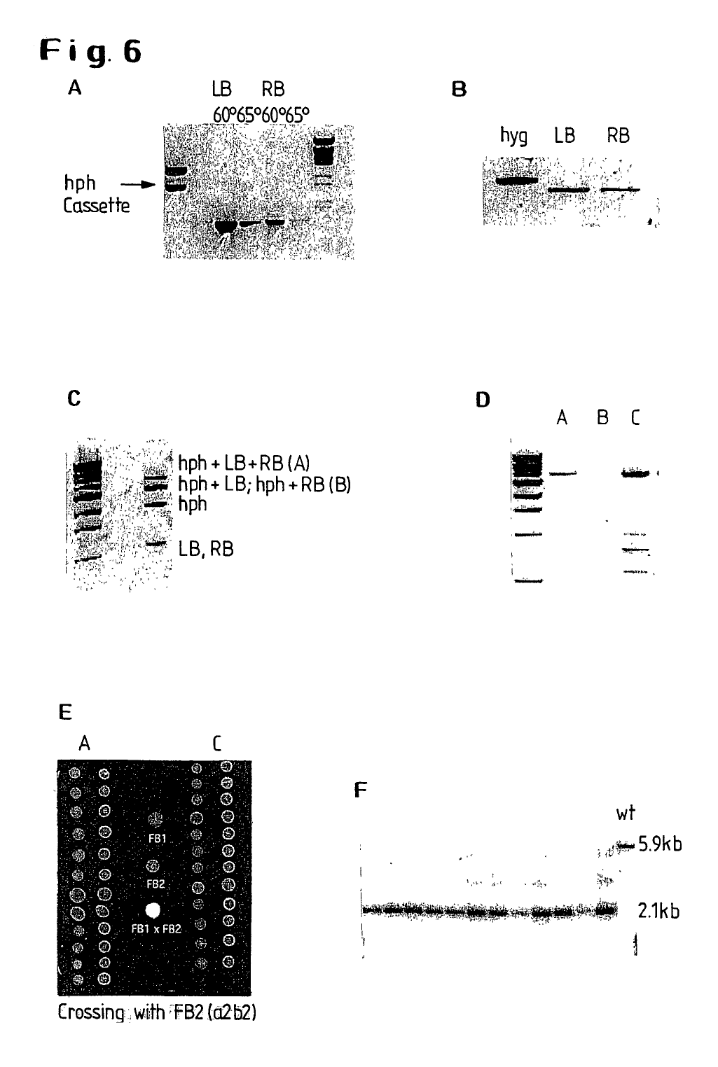

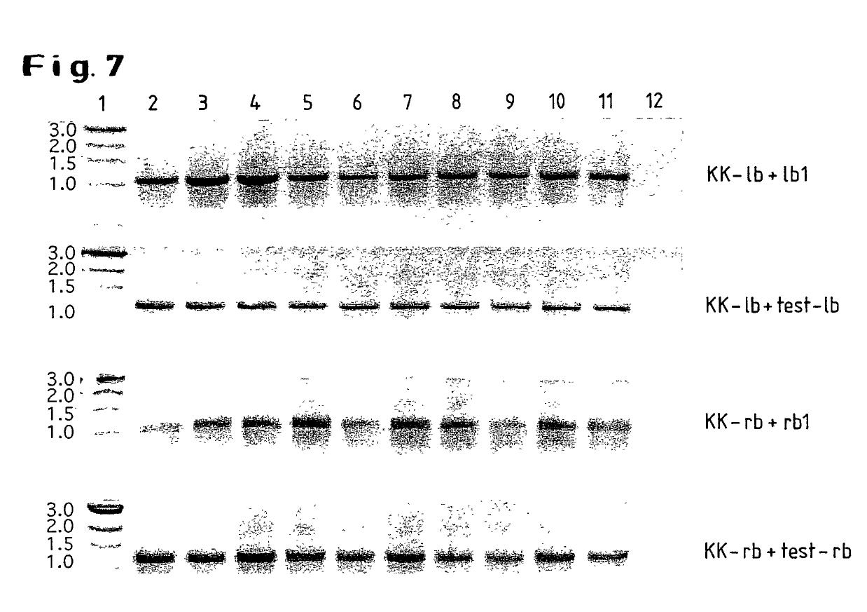

A primer as used herein means a starter oligonucleotide (DNA or RNA, RNA=ribonucleic acid) for the PCR. In this connection, outer primers refer to those primers that are used as starter oligonucleotides for the edges that are relatively distant from the gene of interest (i.e., the gene to be studied). Accordingly, innerprimers refer to those primers that are used as starter oligonucleotides for those regions directly bordering the gene of interest. Written in the 5′-3′ direction, lb denotes the primers for the left border of the gene to be studied and rb denotes the corresponding primers for the right border. According to this definition, the two outer primers are lb1 and rb1 and the two inner primers are lb2 and rb2 as shown in FIG. 1.

A deletion mutant in the present invention means a mutant in which a complete gene has been deleted and/or replaced with a modified gene. Therefore, a knockout mutant as used herein means a deletion mutant in which a gene of interest has been eliminated by deletion and/or gene replacement. The terms deletion mutant and knockout mutant are synonymous and are used interchangeably in this description.

Transformation, in the present invention, is meant to be a simple genetic transfer. The “naked” linear knockout construct penetrates the membrane (and the possibly present cell wall) of a recipient cell without participation of a further supporting substance or support structure and is incorporated into the recipient DNA with the aid of genetic recombination.

Recipient cells, in the present invention, refer to all cells capable of receiving (foreign) DNA via transformation.

A marker gene, in accordance with the present invention, means a gene which provides the transformed cells with a selection advantage (e.g. growth advantage) by expressing the corresponding gene product. Marker genes code, for example, for enzymes causing a resistance to particular antibiotics.

Knockout constructs, as referred to herein, are DNA constructs used for transformation and for preparing the deletion mutants.

A knockout cassette in the present description means a marker gene that includes a promoter and a terminator and which has in both flanking regions specifically constructed cleavage sites for a restriction enzyme.

Normal medium as used herein means a usually known medium for cultivation of the organism studied, such as YEPS medium (see examples) for Ustilago maydis cultures.

A selection medium means a growth medium on which the organism studied can grow only if it is able to express the gene product of the marker gene. An example of a selection medium, which may be mentioned, is YEPS medium which, in addition to the standard components, contains an antibiotic (e.g. hygromycin).

The present invention provides a PCR-based method for preparing deletion mutants involving:

generating the two regions flanking a gene via PCR using primers specific for those regions with two different cleavage sites for the same restriction enzyme being generated at the gene-flanking ends via the two inner primers, lb2 and rb2 (FIG. 1A);

cutting the amplification products with the restriction enzyme specific for the cleavage sites, thereby producing two non-identical overhanging ends (FIG. 1B);

ligating a knockout cassette having the corresponding compatible cleavage sites between the two flanking regions (FIG. 1C) to produce a ligation product;

amplifying the ligation product with PCR using the outer primers lb1 and rb1 (FIG. 1D); and

transforming the amplified ligation product into a recipient cell (FIG. 1E).

Transformation and successful homologous recombination may preferably be checked by whole-cell PCR using primers located outside the flanking regions used for recombination. If primers that are specific for the edge regions of the knockout cassette are used at the same time, an amplicon is obtained only where homologous gene replacement has taken place (FIG. 1F).

Whether the replaced gene is an essential gene may be inferred from the results of various growth experiments. Preference is given to using diploid cells in the method of the present invention, i.e., cells which in each case, have two copies of each gene. Normally, only one copy undergoes homologous recombination.

The growth phenotype may be determined by allowing the organism studied or cells thereof to grow on normal medium (assay 1) followed by growth on selection medium (assay 2) and comparison of the growth behavior results. Where the method of the present invention has eliminated an essential gene, only those organisms in which the gene is still present will grow in assay 1. In contradistinction, no viable organisms would be expected for assay 2.

In haploid organisms it is possible, after meiosis, to assay the meiotic products directly in growth experiments for expression of the resistance gene. Various haploid cells (meiotic products) may be assayed for ability to grow on a normal medium (assay 1). The cells may then be assayed for ability to grow on a medium where growth requires the gene product of the marker gene (assay 2). On this medium, only those organisms in which homologous gene replacement has taken place and the marker gene is present will survive.

In the case of diploid organisms, it is not possible to carry out the assay using the haploid meiotic products directly. If the replaced gene is not an essential gene, 33% of the diploid generation originating from the haploid meiotic products must contain two copies of the resistance gene and, accordingly, no copy of the replaced gene. The resistance gene may be detected according to the description for haploid organisms. However, the loss of the replaced gene may also be documented using PCR. If no cells or organisms containing two copies of the resistance gene are found, the replaced gene is probably an essential gene.

Likewise, it may be possible to utilize the method of the present invention for identifying genes which are important for particular properties (e.g. for pathogenicity) of the cells or organism of interest.

The present invention provides a method for finding essential genes and those genes which are important for particular properties of the cell or organism of interest, involving performing the above-described method and determining the growth phenotype, preferably also as described above.

Essential genes and genes which are important for the pathogenicity of an organism may represent new and interesting targets for the search for new active substances. The present invention provides a method for identifying targets for the search for active substances, in which an essential gene may be identified and expression of that gene prevented or the function of that gene product may be inhibited by exposure to or application of a compound to cells and/or organisms.

Because of the diverse potential uses of the knockout cassette of the present invention, it may be used for preparing deletion mutants in a high throughput process. The present invention therefore provides a method for preparing deletion mutants in a high throughput process.

The description of the method of the present invention for preparing deletion mutants is not to be construed as being limited to a particular cell type. Although it may be possible to use both prokaryotic and eukaryotic cells, preference is given to applying the method to eukaryotic cells, more preferably to fungi, most preferably to Basidiomycota and Ascomycota, in particular to ustilagomycetes such as, for example, Ustilago maydis or hemiascomycetes such as, for example, Saccharomyces cerevisiae. The method of the present invention may be of crucial importance for studying phytopathogenic organisms such as, for example, the smut fungus Ustilago maydis.

All genes for which the flanking regions are known may be suitable for the method of the present invention. The flanking regions amplified by PCR may vary in their size from generally between 60 bp and 2000 bp. The flanking regions may preferably be between 600 bp and 1500 bp, more preferably between 800 bp and 1200 bp, in size.

The specific outer primers lb1 and rb1 employed for the PCR in the method of the present invention may preferably be chosen such that in the genomic context a thymidine (T) is located in each case upstream of the 5′ end of the primer. Because Taq polymerase (DNA polymerase of the thermophilic bacterium Thermus aquaticus) attaches an adenosine (A) at this position during synthesis of the complementary strand, there will be no change in the sequence of the homologous regions. In addition, ligation of the outer ends of the molecules which are limited by primers lb1 and rb1 and are not cut by a restriction endonuclease may be prevented, because those molecules have no phosphate group (due to the primer molecules) and because there are no compatible ends (due to an adenosine (A) which was added by Taq polymerase during synthesis of the complementary strand).

The inner primers lb2 and rb2 employed for the PCR in the method of the present invention may preferably be constructed such that two different cleavage sites for the same restriction enzyme are generated at the ends flanking the gene. Preference may be given to restriction enzymes that have a defined recognition sequence of at least 6 bp (bp=base pairs), more preferably of at least 8 bp, which is interrupted by a variable sequence of preferably from 3 bp to 7 bp, more preferably 3 bp to 5 bp, most preferably 5 bp, and which cut the DNA within the variable sequence such that overhanging ends are produced.

Some examples of suitable restriction enzymes are mentioned herein below. In the sequences given, A=adenine, C=cytosine, G=guanine, T=thymidine and N=any base. Arrows indicate the restriction site.

The enzymes given in Table I, having a recognition sequence of 6 bp and a variable sequence of 3 bp, may be mentioned by way of example only:

TABLE I

| Enzyme | Source organism | Sequence |
|---|---|---|
| AlwN I | Acinetobacter lwoffiiN | 5′ . . . CAGNNN↓CTG . . . 3′ |
| | | 3′ . . . GTC↑NNNGAC . . . 5′ |
| Dra III | Deinococcus radiophilus | 5′ . . . CACNNN↓GTG . . . 3′ |
| | | 3′ . . . GTG↑NNNCAC . . . 5′ |

The enzymes given in Table II, having a recognition sequence of 6 bp and a variable sequence of 5 bp, may be mentioned by way of example only:

TABLE II

| Enzyme | Source organism | Sequence |
|---|---|---|
| AccB7I | Acinetobacter calcoaceticus | 5′ . . . CCANNNN↓NTGG . . . 3′ |
| | B7 | 3′ . . . GGTN↑NNNNACC . . . 5′ |
| Bgl I | Bacillus globigii | 5′ . . . GCCNNNN↓NGGC . . . 3′ |
| | | 3′ . . . CGGN↑NNNNCCG . . . 5′ |
| BstAP I | Bacillus stearothermophilus | 5′ . . . GCANNNN↓NTGC . . . 3′ |
| | AP | 3′ . . . CGTN↑NNNNACG . . . 5′ |
| PflM I | Pseudomonas fluorescens | 5′ . . . CCANNNN↓NTGG . . . 3′ |
| | | 3′ . . . GGTN↑NNNNACC . . . 5′ |

The enzyme given in Table III, having a recognition sequence of 6 bp and a variable sequence of 6 bp, may be mentioned by way of example:

TABLE III

| Enzyme | Source organism | Sequence |
|---|---|---|
| BstX I | Bacillus stearothermophilus | 5′ . . . CCANNNNN↓NTGG . . . 3′ |
| | XI | 3′ . . . GGTN↑NNNNNACC . . . 5′ |

The enzyme given in Table IV, having a recognition sequence of 8 bp and a variable sequence of 5 bp, may be mentioned by way of example:

TABLE IV

| Enzyme | Source organism | Sequence |
|---|---|---|
| Sfi I | Streptomyces fimbriatus | 5′ . . . GGCCNNNN↓NGGCC . . . 3′ |
| | | 3′ . . . CCGGN↑NNNNCCGG . . . 5′ |

Choosing different sequences that are attached to primers lb2 and rb2 to generate the restriction sequence makes it possible to produce two different overhanging ends which are not compatible with one another.

If, for example, the sequences A and B, as shown in Table V, are used as cleavage sites for the restriction enzyme Sfi I, the unequal ends, also shown in Table V, are obtained after restriction:

TABLE V

| | lb2 | rb2 |
|---|---|---|
| Prior to | 5′ . . . GGCCTCTC↓TGGCC . . . 3′ | 5′ . . . GGCCATCT↓AGGCC . . . 3′ |
| restriction | 3′ . . . CCGGT↑GAGTCCGG . . . 5′ | 3′ . . . CCGGT↑AGATCCGG . . . 5′ |
| After | 5′ . . . GGCCTCTC . . . 3′ | 5′ . . . AGGCC . . . 3′ |
| restriction | 3′ . . . CCGGT . . . 5′ | 3′ . . . AGATCCGG . . . 5′ |

The restriction enzymes utilized in the second step of the present invention have already been described herein in the description of primer sequence selection.

The knockout cassette of the third step of the present invention may preferably contain a marker gene for resistance to antibiotics, insecticides, herbicides or fungicides, more preferably a marker gene for resistance to antibiotics, most preferably a marker gene for resistance to the antibiotic hygromycin (e.g. hygromycin phosphotransferase gene, hph), carboxin (e.g. the gene for the iron-sulphur subunit of succinate dehydrogenase, cbx) or phleomycin (phleomycin resistance gene, phleo), preferably a marker gene for resistance to the antibiotic hygromycin.

The knockout cassette of the present invention may preferably be constructed such that it has, after linearization, compatible cleavage sites of the same restriction enzyme at both ends, which have been generated via primers lb2 and rb2 at the flanking ends of the gene to be replaced.

Such construction ensures that only three possible ligation products can be produced:

a knockout cassette containing the left flanking region;

a knockout cassette containing the right flanking region; and

a knockout cassette containing both flanking regions.

The ligation may be carried out using known, commercially available ligases, according to the manufacturer's instructions.

The ligation products from step 3 in the method of the present invention may be amplified in the fourth step, preferably by using the same primers lb1 and rb1 which were used in the first step. In this context it is possible to amplify only those ligation products that have the knockout cassette containing both flanking regions. Thus, a linear DNA fragment may be obtained which contains at both ends the two regions flanking the gene of interest. The marker gene that is connected with the flanking regions on both sides in each case via a restriction cleavage site is located in the center (as shown in the schematic representation of FIG. 2).

The transformation in the method of the present invention may be carried out according to standard techniques of DNA transformation. Examples of generally applicable techniques include, but are not limited to, those for transforming protoplasts, ultrasound techniques, macro- and microinjections, electroporation and density gradient methods. Preference may be given to transforming protoplasts of the fungus Ustilago maydis.

For example, Ustilago maydis protoplasts may be prepared by growing a cell culture to a particular cell density (approx. 5×10⁷, corresponding to an optical density at 600 nm of OD₆₀₀=0.6 to 1.0). The cells may be removed by centrifugation and the pellet resuspended in a sodium citrate/sorbitol buffer (SCS buffer). Following another centrifugation, the pellet may be taken up in SCS buffer containing Novozym. Protoplasts may be formed at room temperature and washed several times with SCS buffer and with a Tris/sorbitol/calcium chloride buffer (STC buffer). The protoplasts may also be stored in this buffer. The transformation may be carried out by mixing the prepared protoplasts with the linear DNA fragment containing the knockout cassette of step D of the present invention. The addition of polyethylene glycol will stimulate DNA transfer into the protoplasts by increasing the permeability of the protoplast membrane (Tsukuda T., Carleton S., Fotheringham S., Holloman W. K. (1988). Isolation and characterization of an autonomously replicating sequence from Ustilago maydis, Mol. Cell. Biol. 8, 3703-3709.) Preference may be given to checking the transformation and successful homologous recombination by using whole-cell PCR. This procedure entails using primers which are preferably complementary to regions outside the flanking gene regions used (e.g. test-lb and test-rb, see FIG. 1F). Combining those test primers with primers for regions within the knockout cassette (KK-lb and KK-rb) results in the corresponding amplicons only in the case of successful gene replacement.

Construction of a Knockout Cassette

The method of the present invention (FIG. 1, step (C)) employs a knockout cassette which may be used to knock out any gene or genes of interest. The knockout cassette of the present invention is constructed such that it is flanked by two different restriction cleavage sites. Therefore, the orientation of the nucleotide sequence may be determined and combination with appropriately prepared flanking regions of any gene becomes possible.

The knockout cassette of the present invention contains a first non-palindromic restriction cleavage site, a marker gene with a promoter and a terminator, and a second non-palindromic restriction cleavage site and, where appropriate, further palindromic restriction cleavage sites.

Furthermore, the present invention provides a method for preparing this knockout cassette, involving:

amplifying a marker gene from plasmid I using primers 1 and 2, thereby introducing different restriction cleavage sites R1 and R2 at both ends of the marker gene, and restricting the PCR product by the enzymes for the the cleavage sites R1 and R2;

linking the PCR product to a terminator by restricting a second plasmid (plasmid II) using the enzymes for the cleavage sites R1 and R2 and replacing the fragment released due to restriction with the PCR fragment containing the marker gene from the preceding step, thereby producing a third plasmid (plasmid III),

amplifying the marker gene starting from plasmid III together with the terminator in a second PCR using primers 1 and 4, thereby generating at least the cleavage site R1 at the 5′ end and of the cleavage site RS-b followed by a cleavage site R4 at the 3′ end, the fragment A obtained may be cloned into a fourth plasmid (plasmid IV), thereby producing a fifth plasmid (plasmid V),

amplifying a functional promoter sequence for the marker gene starting from plasmid I via primers 5 and 6 in a third PCR, thereby generating the cleavage sites R5 and RS-a at the 5′ end of the promoter sequence and at least the cleavage site R1 at the 3′ end, the fragment B obtained may be cloned into plasmid IV, thereby producing a sixth plasmid (plasmid VI),

releasing fragment A by restricting plasmid V using enzymes R1 and R4, releasing fragment B by restricting plasmid VI using enzymes R5 and R1, releasing fragment C by restricting a seventh plasmid (plasmid VII) using the enzymes R5 and R4 and ligating together the released fragments thereby producing an eighth plasmid (plasmid VIII), and

cutting plasmid VIII by the restriction enzyme for cleavage sites RS-a and RS-b.

Plasmid I contains a marker gene and a sequence for a corresponding functional promoter. Plasmid II contains a terminator sequence and a replaceable gene sequence. Plasmid IV is characterized in that particular fragments may be cloned therein by standard methods. An example of a suitable standard method is TOPO cloning using an Invitrogen kit in which plasmid IV is, for example, the plasmid pCR2.1 (Tsukuda T., Carleton S., Fotheringham S., Holloman W. K. (1988). Isolation and characterization of an autonomously replicating sequence from Ustilago maydis, Mol. Cell. Biol. 8, 3703-3709; and manufacturer's information).

Plasmid VII can be cut with the same restriction enzymes for cleavage sites R4 and R5 so that it may be ligated with the corresponding ends of fragments A and B which are preferably released. An example of a suitable vector that may be mentioned in this context is plasmid pBSKSII (Stratagene).

The restriction cleavage sites R1, R2, R4 and R5 may preferably be different palindromic nucleotide sequences which are recognized and cut by different restriction enzymes.

The choice of primers 1 and 6 makes it possible, where appropriate, to simultaneously introduce a plurality of restriction cleavage sites located one behind the other.

For the method for preparing the knockout cassette of the present invention to work properly, the restriction cleavage sites R1 and R2 should be identical to the cleavage sites of plasmid II, which may be used to remove the replaceable gene sequence.

In one embodiment of the present invention, a knockout cassette may be constructed as follows.

A marker gene from plasmid I containing a marker gene (e.g. bacterial hygromycin-resistance gene, hph) and a sequence for a corresponding promoter (e.g. Ustilago maydis hsp70 promoter) (e.g. plasmid pCM54, Tsukuda T., Carleton S., Fotheringham S., Holloman W. K. (1988) Isolation and characterization of an autonomously replicating sequence from Ustilago maydis, Mol. Cell. Biol. 8, 3703-3709) is amplified by PCR using suitable primers 1 and 2 (e.g. primers hph-Nco/Bam and hph-Stop) thereby introducing different restriction cleavage sites R1 (e.g. restriction enzyme Nco I cleavage site having the sequence 5′-C⇓CATGG-3′ at the ATG start codon of the hph gene) and R2 (e.g. restriction enzyme Not I cleavage site having the sequence 5′-GC⇓GGCCGC-3′ at one nucleotide behind the STOP codon of the hph gene) at both ends of the marker gene (FIG. 3A). The PCR product is restricted by the enzymes for cleavage sites R1 (e.g. the enzyme Nco I) and R2 (e.g. the enzyme Not I).

The PCR product containing the marker gene is linked to a terminator. For this purpose, a second suitable plasmid II (e.g. plasmid potefSG (Spellig T., Bottin A., Kahmann R. (1996). Green fluorescent protein (GFP) as a new vital marker in the phytopathogenic fungus Ustilago maydis, Mol. Gen. Genet. 252, 503-509) is used. Plasmid II contains the terminator sequence and a replaceable gene sequence which can be restricted by enzymes for the same cleavage sites R1 and R2. Plasmid II may thus be restricted by enzymes for the cleavage sites R1 (e.g. the enzyme Nco I) and R2 (e.g. the enzyme Not I) and the fragment released due to that restriction (containing, for example, the sGFP gene) may preferably be replaced with the PCR fragment containing the marker gene (hph gene in the example). Due to this cloning, the marker gene is flanked at its 3′ end by the terminator (e.g. agrobacterial NOS terminator of plasmid potefSG, NOS=nopaline synthetase), to ensure efficient termination of the transcription of the marker gene. This produces plasmid III.

A second PCR, starting with plasmid III, amplifies the marker gene together with the terminator. For this purpose, suitable primer 1 (e.g. hph-Nco/Bam) and primer 4 (e.g. 3-hph) are used. This PCR generates at the 5′ end the cleavage sites R3 (e.g. with the sequence 5′-G⇓GATCC-3′ for restriction enzyme BamH I) and R1 (e.g. for the restriction enzyme Nco I) and at the 3′ end the cleavage site RS-b (e.g. for the enzyme Sfi I) followed by a cleavage site R4 (e.g. with the sequence 5′-GAGCT⇓C-3′ for the enzyme Sac I). This produces fragment A, depicted schematically in FIG. 3A. The PCR product (fragment A, FIG. 3A) is cloned directly via a standard method (e.g. TOPO cloning using an Invitrogen kit) into plasmid IV (e.g. the known plasmid pCR2.1), thereby producing plasmid V (e.g. plasmid pBS-hph-Nos).

A third PCR, starting with plasmid I (e.g. plasmid pCM54, see above), amplifies a fragment of a promoter for the marker gene (e.g. a 550 bp fragment of the Ustilago maydis hsp70 promoter) using primers 5 (e.g. primer 5-hsp) and 6 (e.g. primer hsp-Nco). This PCR introduces at the 5′ end of the promoter sequence a cleavage site R5 (e.g. with the sequence 5′-C⇓TCGAG-3′ for restriction enzyme Xho I) and immediately thereafter, the cleavage site RS-a (e.g. for the enzyme Sfi I). At the 3′ end a cleavage site R1 (e.g. for the enzyme Nco I) followed by a restriction site R6 (e.g. with the sequence 5′-A⇓AGCTT-3′ for the enzyme HindIII) is generated. This produces fragment B, depicted schematically in FIG. 3B. The PCR product (fragment B, FIG. 3B) may be cloned directly via a standard method (e.g. TOPO cloning using an Invitrogen kit) into plasmid IV (e.g. the known plasmid pCR2.1) thereby producing plasmid VI (e.g. plasmid pBS-hsp). After cloning, the correct sequences of the fragments generated via PCR may be verified, preferably by sequencing.

Plasmid V may be cut with enzymes R1 and R4, thereby eliminating fragment A (FIG. 3A) containing the marker gene, the terminator sequence and the cleavage site RS-b. Plasmid VI may be cut with enzymes R5 and R1, thereby eliminating fragment B (FIG. 3B) containing the promoter sequence and the cleavage site RS-a. A suitable plasmid VII (e.g. the commercial plasmid pBSKSII, Stratagene) may be cut by R5 and R4, thereby providing fragment C (FIG. 3C). The three fragments obtained may be ligated together, to produce plasmid VIII containing the knockout cassette (schematic representation, see FIG. 4, e.g. plasmid pBS-hhn, see FIG. 5).

The knockout cassette of the present invention may be obtained from plasmid VIII by restriction using the restriction enzyme chosen for cleavage sites RS-a and RS-b and may be used in the method of the present invention for preparing deletion mutants.

A knockout cassette containing the hygromycin phosphotransferase gene (hph) was prepared according to the above-described method. (see examples).

Ustilago maydis

Ustilago maydis is a phytopathogenic Basidiomycete which attacks Zea mays (maize) and the related species Euchlena mexicana (teosinte). This organism provides a model system for studying phytopathogenic development. The method of the present invention may preferably be used in studying the function of genes in this organism.

The first symptoms observed in plants infected by Ustilago maydis may be chloroses and a discoloration caused by anthocyans; followed by the formation of tumors consisting of hypertrophic plant tissue and large amounts of fungal teliospores. The teliospores may be distributed by the wind and thus can readily lead to new infections.

Two morphologically distinct phases in the Ustilago maydis life cycle may be distinguished. Germination of the diploid teliospores leads to the formation of the septate probasidium from whose individual compartments haploid cells, the “sporidia”, are successively removed by abstriction. In this haploid form Ustilago maydis propagates as saprophyte by yeast-like budding. Fusion of two sporidia leads to dramatic morphological and physiological changes: the resulting dikaryon now grows in a filamentous form and is capable of infecting the host plant. After penetration, the fungus grows in the plant initially inter- and intracellularly with branched septate hyphae. Initially, the plant displays no visible defense reactions, however, later, a local massive proliferation of the fungus takes place coinciding with the formation of the plant tumors. The hyphae swell and increasingly wind around one another and at about the same time, karyogamy also takes place. The sporogenous hyphae finally produce the teliospores.

The fusion of the sporidia, which leads to formation of the pathogenic dikaryon, is genetically controlled by the two Ustilago maydis mating-type loci. In order to establish a stable dikaryon, the sporidia must carry different alleles of the biallelic a locus and also different alleles of the multiallelic b locus. Different functions may be assigned to the two loci during development. The a locus controls cell recognition and cell fusion via a pheromone receptor system. Each of the two a alleles encodes a prenylated peptide pheromone (Mfa1 in a1 and Mfa2 in a2) and also a receptor for the pheromone in each other mating type (Pra1 in a1 and Pra2 in a2); the a2 locus additionally contains two genes with unknown function, lga2 and rga2.

After cell fusion a decision is made, via an intracellular self recognition/non-self recognition system encoded by the b locus, about whether the other steps in the sexual and pathogenic development are initiated. The ability to infect the host plant is independent of the a locus; it is sufficient if different alleles of the b locus are present together in a single cell. The b locus thus has the role of the central molecular switch for pathogenesis.

The b locus codes for two different divergently transcribed genes, bE and bW. The proteins encoded by the genes have a derived size of 473 amino acids (bE) and 645 amino acids (bW); the two proteins show no homologies with one another, with the exception of a homeodomain, a domain which is highly conserved in eukaryotes and mediates sequence-specific binding to a DNA. Despite a lack of homology, the proteins have a similar overall structure. In both bE and bW, it is possible to distinguish an amino-terminal variable domain in which the allelic differences between the particular proteins are frequent from a constant highly conserved carboxy-terminal domain. The crossing of mutants carrying deletions in either bE or bW genes shows that, to initiate the pathogenic program, it is sufficient to combine strains having a single functional bE and a single bW gene, as long as the genes originate from different alleles.

The applicability of the method of the present invention was shown by deletion of the b mating-type locus (see examples).

### EXAMPLES

Construction of the Hygromycin Knockout Cassette (hph Cassette)

The bacterial hygromycin-resistance gene (hph) was amplified from plasmid pCM54 (Tsukuda T., Carleton S., Fotheringham S., Holloman W. K. (1988). Isolation and characterization of an autonomously replicating sequence from Ustilago maydis, Mol. Cell. Biol. 8, 3703-3709) by PCR using the primers hph-Nco/Bam (SEQ ID NO. 11) and hph-Stop (SEQ ID NO. 12). The PCR protocol used was according to Innis M. A., Gelfand D. H., Sninsky J. J., White T. J., eds. (1990). PCR Protocols: A Guide to Methods and Applications. (Academic Press, San Diego, USA) cycles:

a) 1 cycle of 10 min at 94° C.,

b) 30 cycles of in each case 1 min at 94° C., 1 min at 60° C., 3 min at 72° C.,

c) 1 cycle of 10 min at 72° C.).

This introduced different restriction cleavage sites in both flanking regions of the hph gene: an Nco I cleavage site having the sequence 5′-C⇓CATGG-3′ at the ATG start codon of the hph gene and an Not I cleavage site having the sequence 5′-GC⇓GGCCGC-3′ at one nucleotide downstream of the STOP codon of the hph gene. The PCR product was restricted by enzymes Nco I and Not I (New England Biolabs, conditions according to the manufacturer's instructions).

The PCR product containing the hph gene was linked to the agrobacterial NOS terminator. For this purpose, the plasmid potefSG (Spellig T., Bottin A., Kahmann R. (1996). Green fluorescent protein (GFP) as a new vital marker in the phytopathogenic fungus Ustilago maydis, Mol. Gen. Genet. 252, 503-509) which contains the sequence of the agrobacterial NOS terminator and a replaceable sequence of the sGFP gene was restricted by enzymes Nco I and Not I (New England Biolabs, conditions according to the manufacturer's instructions). The restriction released the fragment containing the sGFP gene and replaced it with the PCR fragment containing the hph gene. This cloning resulted in the agrobacterial NOS terminator flanking the 3′ and of the hph gene, so as to ensure efficient termination of hph transcription.

In a second PCR, starting from plasmid pCM54, the hph gene was amplified together with the NOS terminator. This entailed using the primers hph-Nco/Bam (SEQ ID NO. 11) and 3-hph (SEQ ID NO. 13). PCR protocol used was again according to Innis et al. cycles:

a) 1 cycle of 10 min at 94° C.,

b) 30 cycles of in each case 1 min at 94° C., 1 min at 60° C., 3 min at 72° C.,

c) 1 cycle of 10 min at 72° C.).

This PCR generated at the 5′ end, the cleavage sites for restriction enzymes BamH I (sequence 5′-G⇓GATCC-3′) and Nco I and at the 3′ end the Sfi I-b cleavage site followed by a cleavage site for the enzyme Sac I (sequence 5′-GAGCT⇓C-3′). The PCR product was cloned directly via TOPO cloning (using an Invitrogen kit, conditions according to the manufacturer's instructions) into plasmid pCR2.1, resulting in plasmid pBS-hph-Nos.

In a third PCR, starting from plasmid pCM54 (see above), a 550 bp fragment of the Ustilago maydis hsp70 promoter was amplified using the primers 5-hsp (SEQ ID NO. 14) and hsp-Nco (SEQ ID NO. 15). PCR protocol used was again according to Innis et al. cycles:

a) 1 cycle of 10 min at 94° C.,

b) 30 cycles of in each case 1 min at 94° C., 1 min at 60° C., 3 min at 72° C.,

c) 1 cycle of 10 min at 72° C.).

This PCR introduced at the 5′ end of the hsp70-promotor sequence a cleavage site for restriction enzyme Xho I (sequence 5′-C⇓TCGAG-3′) followed immediately by the Sfi I-a cleavage site. At the 3′ end a cleavage site for the enzyme Nco I was generated, followed by a cleavage site for the enzyme HindIII (sequence 5′-A⇓AGCTT-3′). The PCR product was cloned directly via a TOPO cloning (using an Invitrogen kit, conditions according to the manufacturer's instructions) into plasmid pCR2.1, producing plasmid pBS-hsp. After cloning, the correct sequences of the fragments generated via PCR were verified by sequencing (ABI 377 automated sequencer, universal and reverse primers, conditions according to the manufacturer's instructions, using the standard universal and reverse primers).

In a last step, i) plasmid pBS-hph-Nos was cut by enzymes Nco I and Sac I (New England Biolabs, conditions according to the manufacturer's instructions), whereby the fragment containing the hph gene, the NOS terminator sequence and the Sfi I-b cleavage site was eliminated, ii) plasmid pBS-hsp was cut by enzymes Xho I and Nco I (New England Biolabs, conditions according to the manufacturer's instructions), whereby the fragment containing the hsp70-promotor sequence and the Sfi I-a cleavage site was eliminated, iii) the commercial plasmid pBSKSII (Stratagene) was cut by Xho l and Sac I (New England Biolabs, conditions according to the manufacturer's instructions), and iv) the three fragments obtained were ligated together (ligase from Roche, conditions according to the manufacturer's instructions), resulting in plasmid pBS-hhn. It was possible to obtain from this plasmid the hygromycin knockout cassette by restriction using Sfi I and to use it in the method of the invention.

FIG. 5 depicts the physical map of plasmid pBS-hhn. The inserted sequence from the Xho I cleavage site via the hsp70 promoter, the hph gene, the NOS terminator to the Sac I cleavage site is the sequence with SEQ ID NO. 16.

Deletion of the Mating Type-b Locus

PCR generated in each case 1 kb regions flanking the b locus, using primers with the sequences lb1 (SEQ ID NO. 1), lb2 (SEQ ID NO. 2), rb1 (SEQ ID NO. 3), and rb2 (SEQ ID NO. 4).

The PCR was carried out under the following conditions: a first denaturation step of 10 min at 94° C. was followed by 30 PCR cycles of in each case 1 min at 94° C. (denaturation), 1 min at 60° C. (hybridization) and 1 min at 72° C. (polymerase reaction), and this was followed by a final incubation at 72° C. for 10 min (PCR protocol according to Innis M. A., Gelfand D. H., Sninsky J. J., White T. J., eds. (1990) PCR Protocols: A Guide to Methods and Applications (Academic Press, San Diego, USA)).

The PCR reaction introduced at the ends of those fragments bordering the b locus two different Sfi I cleavage sites compatible with the Sfi I cleavage site of the hph cassette (FIG. 6A). For this, the primer lb2 contained, in addition to the recognition sequence for the left inner flanking region, the sequence of the Sfi I-b cleavage site and 3 further nucleotides. For this, the primer rb2 contained, in addition to the recognition sequence for the right inner flanking region, the sequence of the Sfi I-a cleavage site and 3 further nucleotides.

The pBS-hhn vector and the PCR generated fragments were restricted correspondingly by Sfi I, purified by gel electrophoresis (FIG. 6B) and ligated with the hph cassette.

After ethanol precipitation, the PCR fragments were restricted by Sfi I (New England Biolabs, 20 Units, 2 h, conditions for the enzyme reaction according to the manufacturer's instructions).

After electrophoretic fractionation in agarose gels, the fragments were purified using the gel elution kit Jetsorb from Genomed according to the manufacturer's instructions.

The flanking regions were ligated with the hph cassette such that 0.2 μg of each flanking region was incubated together with 0.2 μg of the 2 kb hph cassette and 2.5 units of ligase (Roche, conditions for the enzyme reaction according to the manufacturer's instructions).

The non-palindromic different overhangs of the Sfi I cleavage sites prevent ligation of identical as well as different flanking regions. A ligation via the blunt ends of the fragments was ruled out, because the primers used in the PCR reaction were not phosphorylated. FIG. 6C shows that the ligation products met the expectations. In addition to the starting products, two further bands were formed which correspond in size to the hph cassette containing the left flanking region or right flanking region (approx. 3 kb, band B) or to the hph cassette containing both flanking regions (approx. 4 kb, band A).

As FIG. 6D shows, only the product A was amplified in a subsequent PCR using the corresponding outer primers. It was possible to use in this reaction either the ligation product directly (lane C) or, to be safe, the gel-electrophoretically purified product A (lane A). As expected, the product B was not amplified (lane B) (FIG. 6D).

The PCR was carried out under the following conditions: a first denaturation step of 10 min at 94° C. is followed by 30 PCR cycles of in each case 1 min at 94° C. (denaturation), 1 min at 60° C. (hybridization) and 5 min at 72° C. (polymerase reaction), and this was followed by a final incubation at 72° C. for 10 min.

The PCR products from mixtures A and C were precipitated, taken up in water and used directly for transformation of the Ustilago maydis strain FB1 (a1b1) (see below for a description of the transformation). 20 colonies of each transformation mixture were crossed with the compatible strain FB2 (a2b2); in none of the cases were dikaryotic filaments formed which make the colonies appear white (comparable to the control FB1×FB2), pointing to loss of the b function in all of the transformants assayed (FIG. 6E). A Southern blot analysis was carried out for 6 transformants each; in all cases, the b locus had been replaced with the hph cassette (FIG. 6F).

For a quicker check, a PCR analysis may be carried out instead of the Southern analysis. For this purpose, DNA of the transformants was used in a standard PCR mixture (PCR protocol according to Error! Reference source not found.). Instead of the DNA, it was possible to add alternatively between 10⁴ and 10⁵ Ustilago maydis cells (from a freshly grown culture, washed in sterile water) (cycles: a) 1 cycle of 10 min at 94° C., b) 30 cycles of in each case 1 min at 94° C., 1 min at 60° C., 2 min at 72° C., c) 1 cycle of 10 min at 72° C.).

The primer combinations “KK-rb+rb1” and “KK-lb+lb1” should produce PCR products even for ectopic (non-homologous) integration events. The primer combinations “KK-lb+test-lb” and “KK-rb+test-rb” should produce PCR products only in the case of homologous recombination. The primers test-lb and test-rb were located 77 bp and 70 bp, respectively, outside the flanking regions of the knockout cassette used for recombination, which were defined by primers lb1 and rb1.

The PCR reaction using the four primer combinations was carried out for in each case five of the transformants already characterized by Southern analysis and the PCR products were fractionated by gel electrophoresis. As expected, bands of the expected size appeared in all cases (KK-lb+lb1: 1044 bp; KK-lb+test-lb: 1121 bp; KK-rb+rb1: 1062 bp; KK-rb+test-rb: 1132 bp) (see FIG. 7).

Preparation of Ustilago maydis Protoplasts and Transformation

50 ml of a Ustilago maydis culture in YEPS medium were cultivated at 28° C. to a cell density of approx. 5×10⁷/ml (OD₆₀₀0.6 to 1.0). For this purpose, a stationary preculture was diluted in three steps, 1:100, 1:300, 1:1000, and incubated in a culture flask with baffles at 28° C. and 200 rpm for approx. 16 hours. After reaching the desired cell density, the culture was centrifuged at 2500 g for 7 min. The cell pellet was resuspended in 25 ml of SCS buffer and centrifuged again at 2500 g for 7 min. The pellet was resuspended in 2 ml of SCS buffer containing 12.5 mg/ml Novozym 234 (e.g. NovoBiolabs). Protoplast formation was carried out at room temperature and was monitored microscopically every 5 min. The protoplasts were mixed with 10 ml of SCS buffer and centrifuged at 1100 g for 10 min. The supernatant was discarded and the pellet was carefully resuspended three times in in each case 10 ml of SCS buffer and centrifuged. The pellet was washed with 10 ml of STC buffer, resuspended in 500 μl of cold STC buffer and kept on ice.

15 μg of heparin and 50 μl of protoplasts (in STC buffer) were added successively to no more than 10 μl of linear DNA (between 3 μg and 5 μg) and the mixture was cooled on ice for 10 min. 500 μl of PEG3350 [40% (w/w) in STC buffer] was added, carefully mixed with the protoplast suspension and incubated on ice for 15 min.

Transformants were identified by plating out the transformation mixture on agar plates (YEPS medium containing 1.5% agar, 1 M sorbitol and antibiotic; shortly before plating out, this agar layer was overlaid with the same volume of still liquid medium for agar plates which, however, did not contain any antibiotic). The result was determined after incubation at 28° C. for 3 to 4 days.

| YEPS medium (Tsukuda et al.) | | STC buffer | |
|---|---|---|---|
| 1% | yeast extract, | 10 | mM Tris/HCl (pH 7.5), |
| 2% | bactopeptone (Difco), | 1.0 | M sorbitol, |
| 2% | sucrose in water. | 100 | mM CaCl₂ in water. |

SCS buffer

| | |
|---|---|
| 20 | mM sodium citrate, |
| 1.0 | M sorbitol in water, pH 5.8. |

Medium for Agar Plates for Checking Transformation

YEPS medium containing 1.5% agar,

1.0 M sorbitol and antibiotic.

Concentrations Used of Different Antibiotics in the Agar Medium for Checking Transformation:

| | | | | | |
|---|---|---|---|---|---|
| Phleomycin | 80 | μg/ml; | Carboxin | 4 | μg/ml; |
| Hygromycin | 400 | μg/ml; | ClonNAT | 300 | μg/ml. |

The foregoing embodiments of the present invention are offered for the purpose of illustration and not limitation. It will be apparent to those skilled in the art that the embodiments described herein may be modified or revised in various ways without departing from the spirit and scope of the invention. The scope of the invention is to be measured by the appended claims.

What is claimed is:

1. A method for preparing a deletion mutant comprising:
generating two regions flanking a gene by polymerase chain reaction (“PCR”) using primers specific for the regions and generating two different cleavage sites for the same restriction enzyme at the gene-flanking ends via two inner primers lb2 and rb2;
cutting the two flanking regions with the restriction enzyme specific for the cleavage sites, thereby producing two non-identical overhanging ends;
ligating a knockout cassette having corresponding compatible cleavage sites between the two flanking regions to produce a ligation product;
amplifying the ligation product with PCR using outer primers lb1 and rb1; and
transforming the amplified ligation product into a recipient cell.

2. The method of claim 1, wherein eukaryotic cells are used for preparing the deletion mutant.

3. The method of claim 2, wherein the eukaryotic cells comprise fungi.

4. The method of claim 3, wherein the fungi are selected from the group consisting of Basidiomycota and Ascomycota.

5. The method of claim 3, wherein the fungi are selected from the group consisting of, ustilagomycetes and hemiascomycetes.

6. The method of claim 3, wherein the fungi are selected from the group consisting of Ustilago maydis or Saccharomyces cerevisiae.

7. The method of claim 3, wherein the fungi are Ustilago maydis cells.

8. The method of claim 1, wherein the two flanking regions of the gene are each between 60 bp and 2000 bp in size.

9. The method of claim 1, wherein the two flanking regions of the gene are each between 600 bp and 1500 bp in size.

10. The method of claim 1, wherein the two flanking regions of the gene are each between 800 bp and 1200 bp in size.

11. The method of claim 1, wherein two different cleavage sites for the same restriction enzyme are generated via the inner primers lb2 and rb2, wherein the restriction enzyme has a defined recognition sequence of at least 6 bp which is interrupted by a variable restriction sequence of at least 3 bp and is cleaved by the enzyme such that overhanging ends are produced.

12. The method of claim 11, wherein the restriction enzyme has a defined recognition sequence of at least 8 bp which is interrupted by a variable restriction sequence of at least 5 bp.

13. The method of claim 12, wherein the restriction enzyme is Sfi I.

14. The method of claim 13, wherein the two different recognition and restriction cleavage sites have the nucleotide sequences Sfi I-a and Sfi I-b according to SEQ ID NO. 9 and SEQ ID NO. 10 respectively.

15. The method of claim 1, wherein the knockout cassette includes a marker gene.

16. The method of claim 15, wherein the marker gene confers resistance against at least one of an antibiotic, an insecticide, a herbicide and a fungicide.

17. The method of claim 15, wherein the marker gene confers resistance against an antibiotic selected from the group consisting of hygromycin, carboxin and phleomycin.

18. The method of claim 15, wherein the marker gene is the hygromycin phosphotransferase gene.

19. The method of claim 1, wherein protoplasts are used for transformation.

20. The method of claim 1, wherein the deletion mutant is prepared in a high throughput process.

21. A method for finding essential genes comprising:
generating the regions flanking a gene by polymerase chain reaction (“PCR”) using primers specific for the regions and generating two different cleavage sites for the same restriction enzyme at the gene-flanking ends via inner primers lb2 and rb2;
cutting the two flanking regions with the restriction enzyme specific for the cleavage sites, thereby producing two non-identical overhanging ends;
ligating a knockout cassette having corresponding compatible cleavage sites between the two flanking regions to produce a ligation product;
amplifying the ligation product with PCR using outer primers lb1 and rb1;
transforming the amplified ligation product into a recipient cell; and determining the growth phenotype.

22. The method of claim 21, wherein determining the growth phenotype further includes:
growing the recipient cell on normal medium (assay 1);
growing the recipient cell on selection medium (assay 2); and
comparing the growth from assay1 and assay 2.

23. A method for identifying targets for the search for active substances, comprising identifying an essential gene by the method of claim 21 and preventing expression of the gene or inhibiting the function of the gene product by exposing the cell to at least one compound.

24. A knockout cassette comprising:
a first non-palindromic restriction cleavage site;
a marker gene with a promoter and a terminator; and
a second non-palindromic restriction cleavage site.

25. The knockout cassette of claim 24, wherein the non-palindromic restriction cleavage sites have the nucleotide sequences according to SEQ ID NO. 9 and SEQ ID NO. 10.

26. The knockout cassette of claim 24, wherein the marker gene is the hygromycin phosphotransferase gene.

27. The knockout cassette of claim 24, wherein the promoter is the Ustilago maydis hsp70 promoter.

28. The knockout cassette of claim 24, wherein the terminator is the agrobacterial NOS terminator.

29. The knockout cassette of any one of claims 24 to 28 having the nucleotide sequence according to SEQ ID NO. 16.

30. A method for preparing a knockout cassette comprising:
amplifying a marker gene from a first plasmid (plasmid 1) by PCR using primers 1 and 2, thereby introducing different restriction cleavage sites R1 and R2 at both ends of the marker gene to produce a PCR product;
restricting the PCR product by the enzymes for the cleavage sites R1 and R2 to produce a PCR fragment containing the marker gene;
linking the restricted PCR product to a terminator by restricting a second plasmid (plasmid II) using the enzymes for the cleavage sites R1 and R2 and replacing the fragment released with the PCR fragment containing the marker gene producing a third plasmid (plasmid III);
amplifying the marker gene and the terminator in plasmid III by PCR using primers 1 and 4, thereby generating fragment A containing at least the cleavage site R1 at the 5′ end and of the cleavage site RS-b followed by a cleavage site R4 at the 3′ end;
cloning fragment A into a fourth plasmid (plasmid IV), thereby producing a fifth plasmid (plasmid V);
amplifying a functional promoter sequence for the marker gene from plasmid I by PCR with primers 5 and 6 thereby generating fragment B containing the cleavage sites R5 and RS-a at the 5′ end of the promoter sequence and at least the cleavage site R1 at the 3′ end;
cloning fragment B into plasmid IV, thereby producing a sixth plasmid (plasmid VI);
releasing the fragment A by restricting the plasmid V using the enzymes R1 and R4;
releasing the fragment B by restricting the plasmid VI using the enzymes R5 and R1;
releasing a fragment C by restricting a seventh plasmid (plasmid VII) using the enzymes R5 and R4;
ligating together the fragments A, B and C, thereby producing an eighth plasmid (plasmid VIII); and
cutting plasmid VIII by the restriction enzyme for cleavage sites RS-a and RS-b.


SEQUENCE LISTING (16 sequences)

SEQ ID NO: 1 - DNA, 24 bp, Primer lb1
gaagctagcggaaaagtgggtgag

SEQ ID NO: 2 - DNA, 41 bp, Primer lb2
cacggcctgagtggccgactttgaaagcgcccactggatgg

SEQ ID NO: 3 - DNA, 23 bp, Primer rb1
ccgtagttgtgcgagagcagagg

SEQ ID NO: 4 - DNA, 40 bp, Primer rb2
gtgggccatctaggccgatttcagtgatacgtttagtccc

SEQ ID NO: 5 - DNA, 21 bp, Primer KK-lb
actagatccgatgataagctg

SEQ ID NO: 6 - DNA, 20 bp, Primer KK-rb
aactcgctggtagttaccac

SEQ ID NO: 7 - DNA, 22 bp, Primer test-lb
gtggaagatgctggaccgaagc

SEQ ID NO: 8 - DNA, 22 bp, Primer test-rb
accgtgcaactcatcttcggtg

SEQ ID NO: 9 - DNA, 13 bp, SfiI-a (pBS-hhn)
ggcctagatggcc

SEQ ID NO: 10 - DNA, 13 bp, SfiI-b (pBS-hhn)
ggcctgagtggcc

SEQ ID NO: 11 - DNA, 25 bp, Primer hph-Nco/Bam
gcggatcccatggaaaagcctgaac

SEQ ID NO: 12 - DNA, 32 bp, Primer hph-stop
gggcggccgctctattcctttgccctcggacg

SEQ ID NO: 13 - DNA, 44 bp, Primer 3-hph
cggagctcggccactcaggcctcatgtttgacagcttatcatcg

SEQ ID NO: 14 - DNA, 45 bp, Primer 5-hsp
cgctcgaggcctagatggccgaacgtggtaactaccagcgagttc

SEQ ID NO: 15 - DNA, 35 bp, Primer hsp-Nco
cggaagcttccatggccccccgggatctggcaacg

SEQ ID NO: 16 - DNA, 1908 bp, XhoI/SacI-Insert (pBS-hhn)
ctcgaggcctagatggccgaacgtggtaactaccagcgagttctgcaaacttcaaaaaaaaaatctgggcacgatgaaagttgagctaacgctgacgctcacaaatggcgtggctaaaggaagcgagacaatcggaaaattgttctctcgggcaccacaaagctgttgttagtcgctgaagaacaattccaactgattccgccgccttcctattgcgtcagccttgtacctaagctgccgagtaacgtcactcaacctctcttttcagactgctttgctccgcgaatacttttcttctatgcgctcaagaaaatgacacagcacaccaagctctgcaaactttcttcgctaatctgacgcgaaatgtgagccatttcttctcgcctgcaatggcaatgcgtctgtgcggcgatgagaatcacgatgcggaatgggtggctggaagttcatagagatgctgagttgttggagcgacatggtacataagcatgagtctgtcctgatttccaccctcccgtctttcatcaactttctcgtctgacccttccgttgccagatcccggggggccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagcggccgcccggctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccgatgataagctgtcaaacatgaggcctgagtggccgagctc